SEQUENCE LISTING (23 sequences)

SEQ ID NO: 1 - protein, 417 aa, Homo sapiens
MSSKYPRSVRRCLPLWALTLEAALILLFYFFTHYDASLEDQKGLVASYQVGQDLTVMAAIGLGFLTSSFRRHSWSSVAFNLFMLALGVQWAILLDGFLSQFPSGKVVITLFSIRLATMSALSVLISVDAVLGKVNLAQLVVMVLVEVTALGNLRMVISNIFNTDYHMNMMHIYVFAAYFGLSVAWCLPKPLPEGTEDKDQTATIPSLSAMLGALFLWMFWPSFNSALLRSPIERKNAVFNTYYAVAVSVVTAISGSSLAHPQGKISKTYVHSAVLAGGVAVGTSCHLIPSPWLAMVLGLVAGLISVGGAKYLPGCCNRVLGIPHSSIMGYNFSLLGLLGEIIYIVLLVLDTVGAGNGMIGFQVLLSIGELSLAIVIALMSGLLTGLLLNLKIWKAPHEAKYFDDQVFWKFPHLAVGF

SEQ ID NO: 2 - DNA, 1254 bp, Artificial Sequence
  Synthetic
atgagctctaagtacccgcggtctgtccggcgctgcctgcccctctgggccctaacactggaagcagctctcattctcctcttctatttttttacccactatgacgcttccttagaggatcaaaaggggctcgtggcatcctatcaagttggccaagatctgaccgtgatggcggccattggcttgggcttcctcacctcgagtttccggagacacagctggagcagtgtggccttcaacctcttcatgctggcgcttggtgtgcagtgggcaatcctgctggacggcttcctgagccagttcccttctgggaaggtggtcatcacactgttcagtattcggctggccaccatgagtgctttgtcggtgctgatctcagtggatgctgtcttggggaaggtcaacttggcgcagttggtggtgatggtgctggtggaggtgacagctttaggcaacctgaggatggtcatcagtaatatcttcaacacagactaccacatgaacatgatgcacatctacgtgttcgcagcctattttgggctgtctgtggcctggtgcctgccaaagcctctacccgagggaacggaggataaagatcagacagcaacgatacccagtttgtctgccatgctgggcgccctcttcttgtggatgttctggccaagtttcaactctgctctgctgagaagtccaatcgaaaggaagaatgccgtgttcaacacctactatgctgtagcagtcagcgtggtgacagccatctcagggtcatccttggctcacccccaagggaagatcagcaagacttatgtgcacagtgcggtgttggcaggaggcgtggctgtgggtacctcgtgtcacctgatcccttctccgtggcttgccatggtgctgggtcttgtggctgggctgatctccgtcgggggagccaagtacctgccggggtgttgtaaccgagtgctggggattccccacagctccatcatgggctacaacttcagcttgctgggtctgcttggagagatcatctacattgtgctgctggtgcttgataccgtcggagccggcaatggcatgattggcttccaggtcctcctcagcattggggaactcagcttggccatcgtgatagctctcatgtctggtctcctgacaggtttgctcctaaatcttaaaatatggaaagcacctcatgaggctaaatattttgatgaccaagttttctggaagtttcctcatttggctgttggattttaa

SEQ ID NO: 3 - protein, 417 aa, Homo sapiens [sequence omitted]

SEQ ID NO: 4 - DNA, 1254 bp, Artificial Sequence
  Synthetic
atgagctctaagtacccgcggtctgtccggcgctgcctgcccctctgcgccctaacactggaagcagctctcattctcctcttctatttttttacccactatgacgcttccttagaggatcaaaaggggctcgtggcatcctatcaagtcggccaagatctgaccgtgatggcggcccttggcttgggcttcctcacctcaaatttccggagacacagctggagcagtgtggccttcaacctcttcatgctggcgcttggtgtgcagtgggcaatcctgctggacggcttcctgagccagttccctcctgggaaggtggtcatcacactgttcagtattcggctggccaccatgagtgctatgtcggtgctgatctcagcgggtgctgtcttggggaaggtcaacttggcgcagttggtggtgatggtgctggtggaggtgacagctttaggcaccctgaggatggtcatcagtaatatcttcaacacagactaccacatgaacctgaggcacttctacgtgttcgcagcctattttgggctgactgtggcctggtgcctgccaaagcctctacccaagggaacggaggataatgatcagagagcaacgatacccagtttgtctgccatgctgggcgccctcttcttgtggatgttctggccaagtgtcaactctgctctgctgagaagtccaatccaaaggaagaatgccatgttcaacacctactatgctctagcagtcagtgtggtgacagccatctcagggtcatccttggctcacccccaaaggaagatcagcatgacttatgtgcacagtgcggtgttggcaggaggcgtggctgtgggtacctcgtgtcacctgatcccttctccgtggcttgccatggtgctgggtcttgtggctgggctgatctccatcgggggagccaagtgcctgccggtgtgttgtaaccgagtgctggggattcaccacatctccgtcatgcactccatcttcagcttgctgggtctgcttggagagatcacctacattgtgctgctggtgcttcatactgtctggaacggcaatggcatgattggcttccaggtcctcctcagcattggggaactcagcttggccatcgtgatagctctcacgtctggtctcctgacaggtttgctcctaaatctcaaaatatggaaagcacctcatgtggctaaatattttgatgaccaagttttctggaagtttcctcatttggctgttggattttaa

SEQ ID NO: 5 - protein, 23 aa, Homo sapiens
HYDASLEDQKGLVASYQVGQDLT

SEQ ID NO: 6 - protein, 15 aa, Homo sapiens
FPSGKVVITLFSIRL

SEQ ID NO: 7 - protein, 14 aa, Homo sapiens
NIFNTDYHMNMMHI

SEQ ID NO: 8 - protein, 21 aa, Homo sapiens
WPSFNSALLRSPIERKNAVFN

SEQ ID NO: 9 - protein, 6 aa, Homo sapiens
CHLIPS

SEQ ID NO: 10 - protein, 11 aa, Homo sapiens
VLDTVGAGNGM

SEQ ID NO: 11 - protein, 15 aa, Homo sapiens
FPPGKVVITLFSIRL

SEQ ID NO: 12 - protein, 14 aa, Homo sapiens
NIFNTDYHMNLRHF

SEQ ID NO: 13 - protein, 21 aa, Homo sapiens
WPSVNSALLRSPIQRKNAMFN

SEQ ID NO: 14 - protein, 21 aa, Homo sapiens
WPSVNSPLLRSPIQRKNAMFN

SEQ ID NO: 15 - protein, 11 aa, Homo sapiens
VLHTVWNGNGM

SEQ ID NO: 16 - protein, 21 aa, Homo sapiens
MALPVTALLLPLALLLHAARP

SEQ ID NO: 17 - protein, 21 aa, Homo sapiens
IYIWAPLAGTCGVLLLSLVIT

SEQ ID NO: 18 - protein, 45 aa, Homo sapiens
TTTPAPRPPTPAPTIASQPLSLRPEACRPAAGGAVHTRGLDFACD

SEQ ID NO: 19 - protein, 12 aa, Artificial Sequence
  Synthetic
GGGGSGGGGSSG

SEQ ID NO: 20 - protein, 42 aa, Homo sapiens
KRGRKKLLYIFKQPFMRPVQTTQEEDGCSCRFPEEEEGGCEL

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 917 aa, Artificial Sequence
  Synthetic
MSSKYPRSVRRCLPLWALTLEAALILLFYFFTHYDASLEDQKGLVASYQVGQDLTVMAAIGLGFLTSSFRRHSWSSVAFNLFMLALGVQWAILLDGFLSQFPSGKVVITLFSIRLATMSALSVLISVDAVLGKVNLAQLVVMVLVEVTALGNLRMVISNIFNTDYHMNMMHIYVFAAYFGLSVAWCLPKPLPEGTEDKDQTATIPSLSAMLGALFLWMFWPSFNSALLRSPIERKNAVFNTYYAVAVSVVTAISGSSLAHPQGKISKTYVHSAVLAGGVAVGTSCHLIPSPWLAMVLGLVAGLISVGGAKYLPGCCNRVLGIPHSSIMGYNFSLLGLLGEIIYIVLLVLDTVGAGNGMIIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 23 - protein, 694 aa, Artificial Sequence
  Synthetic
MALPVTALLLPLALLLHAARPHYDASLEDQKGLVASYQVGQDLTKKFPSGKVVITLFSIRLKKNIFNTDYHMNMMHIKKWPSFNSALLRSPIERKNAVFNKKCHLIPSKKVLDTVGAGNGMIASGGGGSGGGGSSGIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

(19) United States
(12) Patent Application Publication
(10) Pub. No.: US 2023/0270782 A1
(43) Pub. Date: Aug. 31, 2023

(54) COMPOSITIONS AND METHODS TO TARGET ANTI-RH ANTIBODY

(71) Applicant: PORTON ADVANCED SOLUTIONS LTD., Suzhou, Jiangsu (CN)

(72) Inventors: Lingjie KONG, Union City, CA (US); Shicheng ZHU, Suzhou, Jiangsu (CN); Jean WANG, Union City, CA (US)

(21) Appl. No.: 18/043,574

(22) PCT Filed: Sep. 3, 2021
(86) PCT No.: PCT/CN2021/116366

§ 371 Date: Feb. 28, 2023

(30) Foreign Application Priority Data
Sep. 3, 2020 (WO) PCT/CN2020/113184

(51) Int. Cl. A61K 35/17; C07K 14/705; C07K 14/725; C12N 15/86; C12N 5/0783; A61P 7/00

(52) U.S. Cl. CPC A61K 35/17; C07K 14/70517; C07K 14/70578; C07K 14/7051; C12N 15/86; C12N 5/0636; A61P 7/00; A61K 2039/5156

(57) ABSTRACT

Provided is a chimeric alloantibody receptor (CALAR) specific for alloantibody-based B cell receptor (BCR), wherein the alloantibody is specific for an Rh factor. Also provided are compositions comprising the CALAR, polynucleotides encoding the CALAR, vectors comprising a polynucleotide encoding the CALAR, engineered cells comprising the CALAR, and method using the same.

### CROSS-REFERENCE TO RELATED APPLICATIONS

This application claims priority PCT application no. PCT/CN2020/113184, filed Sep. 3, 2020, the disclosure of which is incorporated herein by reference.

### FIELD OF THE INVENTION

The present disclosure generally relates to therapeutics including treatment with immunosuppressive medication. In particular, the present disclosure relates to compositions and methods for treating or preventing a disorder associated with anti-Rh antibody.

### BACKGROUND

The Rh blood group system is one of the most polymorphic and immunogenic systems known in humans. The Rh system include a large number of Rh antigens, and Rh incompatibility can cause serious complication for the fetus of a woman who is alloimmunized to Rh by pregnancy or transfusion. A woman carrying an Rh incompatible fetus is at the risk of producing antibodies against Rh factor (anti-Rh antibodies) when her blood contacts with the fetus blood during events such as birth-giving, miscarriage, induced abortion, ectopic pregnancy or chorionic villus sampling. Such maternal derived anti-Rh antibody may have minimal impact at the first pregnancy. However, in subsequent pregnancy of the next Rh incompatible fetus, the maternal derived anti-Rh antibody will cross the placental barrier through passive immune transfer and attack the fetus erythrocytes, resulting in hemolytic disease of the newborn (NDH). Among the various Rh antigens, RhD antigen (or D antigen) accounts for the majority of the maternal alloimmulization. NDH due to RhD antigen incompatibility is prevalent in Caucasians, who have the highest incidence of the Rh D negative phenotype (about 15%), but is less common in blacks and Asians.

To prevent the maternal alloimmunization to Rh factor and the generation of anti-Rh antibody, a woman can be injected with prophylactic anti-Rh immunoglobulin (such as anti-RhD immunoglobulin) at any event when the woman may become alloimmunized to Rh antigen. However, once the anti-Rh antibody is formed, such preventive measure is not helpful, and currently there is no treatment to prevent the attack of maternal anti-Rh antibody on the fetus erythrocyte. Therefore, a need exists for novel and effective treatment for maternal anti-Rh antibody caused disorders.

### SUMMARY OF INVENTION

The present disclosure in one aspect provides a polynucleotide encoding a chimeric alloantibody receptor (CALAR). In some embodiments, the CALAR comprises an extracellular domain comprising an immunogenic fragment of Rh factor, a transmembrane domain and an intracellular signaling domain, wherein the extracellular domain binds to a B cell receptor (BCR) to Rh antigen expressed on a B-cell, wherein a cell expressing the CALAR binds the BCR expressed on the B-cell or induces killing of the B-cell expressing the BCR.

In some embodiments, the immunogenic fragment of Rh factor comprises an immunogenic fragment of Rh D factor. In some embodiments, the immunogenic fragment of Rh factor comprises a sequence selected from the group listed in Table 2 or a sequence having at least 90% identity thereto, or a sequence having 1, 2, 3, 4 or 5 amino acid residues difference therefrom.

In some embodiments, the CALAR further comprises a signal peptide. In some embodiments, the signal peptide comprises the signal peptide of CD8 alpha. In some embodiments, the signal peptide of CD8 alpha comprises the sequence of SEQ ID NO: 16 or a sequence having at least 90% identity thereto; or a sequence having 1, 2, 3, 4, 5 amino acid residue difference therefrom.

In some embodiments, the transmembrane domain comprises a transmembrane domain of CD8 alpha. In some embodiments, the transmembrane domain of CD8 alpha comprises the sequence of SEQ ID NO: 17 or a sequence having at least 90% identity thereto; or a sequence having 1, 2, 3, 4, 5 amino acid residue difference therefrom.

In some embodiments, the extracellular domain is linked to the transmembrane domain by a hinge region. In some embodiments, the hinge region comprises a hinge region of CD8 alpha or a GS linker. In some embodiments, the hinge region of CD8 alpha comprises the sequence of SEQ ID NO: 18 or a sequence having at least 90% identity thereto; or a sequence having 1, 2, 3, 4, 5 amino acid residue difference therefrom. In some embodiments, the GS linker comprises the sequence of SEQ ID NO: 19 or a sequence having at least 90% identity thereto; or a sequence having 1, 2, 3, 4, 5 amino acid residue difference therefrom.

In some embodiments, the intracellular domain comprises a costimulatory domain and a signaling domain. In some embodiments, the costimulatory domain comprises an intracellular domain of CD137. In some embodiments, the intracellular domain of CD137 comprises the sequence of SEQ ID NO: 20 or a sequence having at least 90% identity thereto; or a sequence having 1, 2, 3, 4, 5 amino acid residue difference therefrom. In some embodiments, the intracellular domain comprises a signaling domain of CD3 zeta. In some embodiments, the signaling domain of CD3 zeta comprises the sequence of SEQ ID NO: 21 or a sequence having at least 90% identity thereto; or a sequence having 1, 2, 3, 4, 5 amino acid residue difference therefrom.

In another aspect, the present disclosure provides a polypeptide encoded by the polynucleotide described herein.

In another aspect, the present disclosure provides a vector comprising the polynucleotide described herein, wherein the polynucleotide encoding the CALAR is operatively linked to at least one regulatory polynucleotide element for expression of the CALAR. In some embodiments, the vector is a plasmid vector, a viral vector, a retrotransposon, a site directed insertion vector, or a suicide expression vector. In some embodiments, the vector is a lentiviral vector, a retroviral vector or an AAV vector.

In another aspect, the present disclosure provides an engineered cell comprising the polynucleotide described herein. In some embodiments, the engineered cell is a T cell or an NK cell.

In another aspect, the present disclosure provides a method of treating or preventing a disorder associated with anti-Rh antibody. In some embodiments, the method comprises administering an effective amount of the engineered cell described herein in a subject in need thereof. In some embodiments, the disorder associated with anti-Rh antibody is hemolytic disease of the newborn. In some embodiments, the engineered cell is an autologous cell. In some embodiments, the engineered cell is an allogeneic cell. In some embodiments, the method further comprises co-administering an agent that increases the efficacy of the engineered cells. In some embodiments, the method further comprises co-administering an agent that ameliorates side effects associated with the administration of the engineered cells.

### BRIEF DESCRIPTION OF THE DRAWINGS

The accompanying drawings, which are incorporated herein, form part of the specification. Together with this written description, the drawings further serve to explain the principles of, and to enable a person skilled in the relevant art(s), to make and use the present disclosure.

FIG. 1 illustrates that chimeric alloantibody receptor (CALAR) expressed on engineered T cells targets B-cell receptor (BCR) expressed on certain B cells that are specific for an Rh antigen.

FIG. 2 illustrates a schematic diagram of an exemplary CALAR construct.

FIG. 3 illustrates the validation of the K562-R593 target cells. The surface IgG BCRs were confirmed by anti-human CD79b antibody and anti-human kappa light chain antibody.

FIG. 4 illustrates the efficacy of RHD CALAR T cells in killing K562-R593 target cells. K562 cells are non-target cells used as control. NT refers to T cells not transduced by lentivirus expressing RHD.

FIG. 5 illustrates INF-γ production of RHD CALAR T cells after 20 hours of co-culture with K562-R593 target cells. INF-γ concentration in co-cultures of RHD1 or RHD2 CALAR-T cells with K562-R593 target cells increased compared to T cells only, NT, or K562 controls. NT refers to T cells that are not transduced by lentivirus expressing RHD.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definition

The following definitions are provided to assist the reader. Unless otherwise defined, all terms of art, notations and other scientific or medical terms or terminology used herein are intended to have the meanings commonly understood by those of skill in the art. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

“Antigen” refers to a molecule that provokes an immune response. This immune response may be either humoral, or cell-mediated response, or both. The skilled artisan will understand that any macromolecule, including virtually all proteins or peptides, can serve as an antigen. It is readily apparent that the present disclosure includes alloantigens acting as antigen eliciting immune response.

“Antibody” refers to a polypeptide of the immunoglobulin (Ig) family that binds with an antigen. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region (abbreviated herein as CL). The light chain constant region is comprised of one domain. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen.

“Monoclonal antibody” refers to an antibody that is made by identical immune cells that are all clones of a unique parent cell.

“Alloantigen” refers to any nonself antigen that presents in only some individuals of a species and stimulates the immune responses in those individuals of the species who lack the antigen. Alloantigen is resulted from the polymorphism of the genes encoding such antigen. Examples of alloantigens include, but are not limited to, blood group antigens (e.g., Rh antigens) and histocompatibility antigens

“Alloantibody” refers to an antibody specific for an alloantigen.

“Alloimmunization” is an immune response, either cell-mediated or antibody-mediated response, to an alloantigen. Maternal alloimmunization occurs when a woman's immune system is sensitized to fetus blood group antigens (e.g., Rh antigens), stimulating the production of alloantibodies.

“Rh factor” refers to an erythroid protein encoded by RH genes. Rh factors include RhD factor, encoded by RHD gene, and RhCE factor, encoded by RHCE gene. The RHD and RHCE genes are located in close proximity in an inverted orientation on chromosome 1. RhD and RhCE factors, both having 12 transmembrane spans with the N-terminus and C-terminus oriented to the cytoplasm, differ by 32-35 of 416 amino acid residues (Westhoff C M, Semin Hematol., 2007, 44: 42-50). As used herein, Rh factor is intended to encompass both wild-type and variants that have minimal effect on the recognition by an anti-Rh antibody. In some embodiments, the polynucleotide encoding Rh factor is codon optimized.

“Rh antigen” refers to a collection of epitopes along the Rh factor. RHD and RHCE genes frequently carry point mutations or have rearrangements and exchanges, resulting in a great number of Rh antigens. At least 49 distinct Rh antigens have been identified, and D, C, E, c, and e are among the most significant. The D antigen is carried by RhD factor, and the C or c antigen together with either E or e antigen is carried by RhCE factor. Alloimmunization elicited by D and c antigen can cause severe diseases, while that by C, E, and e antigen can cause mild to moderate disease. D, C, E, c, and e antigens, respectively, have frequencies of 85%, 68%, 29%, 80% and 98% in Caucasians, 92%, 27%, 22%, 96% and 98% in blacks, and 99%, 93%, 39%, 47% and 96% in Asians. The Rh D-negative phenotype are most prevalent in Caucasians (15%), less common in blacks (8%), and rare in Asians (1%) (Reid ME and Lomas-Francis C. The Blood Group Antigen Facts Book. Second ed. 2004, New York: Elsevier Academic Press).

“Autologous” cells refer to any cells derived from the same subject into which are later to be re-introduced.

“Allogeneic” cells refer to any cells derived from a different subject of the same species.

“B-cell receptor” or “BCR” refers to a transmembrane immunoglobulin molecule on the surface of B cell that recognize a specific antigen.

“Chimeric alloantibody receptor” or “CALAR” refers to an engineered receptor that is capable of grafting a desired specificity to an alloantibody or a B-cell receptor corresponding to the alloantibody into immune cells capable of cell-mediated cytotoxicity. Typically, a CALAR is a fusion polypeptide comprises an extracellular domain that introduces the desired specificity, a transmembrane domain and an intracellular domain that transmits a signal to the immune cells when the immune cells bind to the alloantibody or the B-cell receptor.

“Co-stimulatory ligand” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with a major histocompatibility complex (MEW) molecule loaded with peptide, mediates a T cell response, including, but not limited to, proliferation, activation, differentiation, and the like.

“Co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to CD28 and 4-1-BB.

“Effector cells” used in the context of immune cells refers to cells that can be activated to carry out effector functions in response to stimulation. Effector cells may include, without limitation, NK cells, cytotoxic T cells and helper T cells.

“Effective amount” or “therapeutically effective amount” refers to an amount of cells, composition, formulation or any material as described here effective to achieve a desirable biological result. Such results may include, without limitation, elimination of B cells expressing a specific BCR and the antibodies produced therefrom.

“Epitope” or “immunogenic fragment” or “antigenic determinant” refers to a portion of an antigen recognized by an antibody or an antigen-binding fragment thereof. An epitope can be linear or conformational.

Percentage of “identity” or “sequence identity” in the context of polypeptide or polynucleotide is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Operatively linked” refers to a functional relationship between two or more polynucleotide sequences. In the context of a polynucleotide encoding a fusion protein, such as a polypeptide chain of a CALAR of the disclosure, the term means that the two or more polynucleotide sequences are joined such that the amino acid sequences encoded by these segments remain in-frame. In the context of transcriptional or translational regulation, the term refers to the functional relationship of a regulatory sequence to a coding sequence, for example, a promoter in the correct location and orientation to the coding sequence so as to modulate the transcription.

“Immunogenicity” or “immunogenic” refers to the ability of a foreign substance, such as an antigen, to provoke an immune response in the body of a subject. The immunogenic response typically includes both cell-mediated and antibody-mediated immune response. As used in the context of an autoantigen, an “immunogenic fragment” refers to a region of the autoantigen that elicit the immune response of the host. Such response can lead to the production of autoantibodies against the autoantigen and cause autoimmune diseases.

“Polynucleotide” or “nucleic acid” refers to a chain of nucleotides. As used herein polynucleotides include all polynucleotide sequences which are obtained by any means available in the art, including, without limitation, recombinant means and synthetic means.

“Polypeptide,” and “protein” are used interchangeably, and refer to a chain of amino acid residues covalently linked by peptide bonds. The polypeptides include natural peptides, recombinant peptides, synthetic peptides, or a combination thereof.

“T cell receptor” or “TCR” refers to a protein complex on the surface of T cells that is responsible for recognizing fragments of antigen as peptides bound to MHC molecules.

“Vector” refers to a vehicle into which a polynucleotide may be operably inserted so as to deliver, replicate or express the polynucleotide. A vector may contain a variety of regulatory elements including, without limitation, origin of replication, promoter, transcription initiation sequences, enhancer, selectable marker genes, and reporter genes. A vector may also include materials to aid in its entry into a host cell, including but not limited to a viral particle, a liposome, or ionic or amphiphilic compounds.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

### Chimeric Alloantibody Receptor

The Rh blood group system is one of the most polymorphic and immunogenic systems known in humans. The Rh system include a large number of Rh antigens, and Rh incompatibility can cause serious complication for the fetus of a woman who is alloimmunized to Rh by pregnancy or transfusion. The maternal derived anti-Rh antibody can cross the placental barrier through passive immune transfer and attack the fetus erythrocytes, resulting in hemolytic disease of the newborn (NDH). Among the various Rh antigens, RhD antigen accounts for over 50% of the maternal alloimmulization.

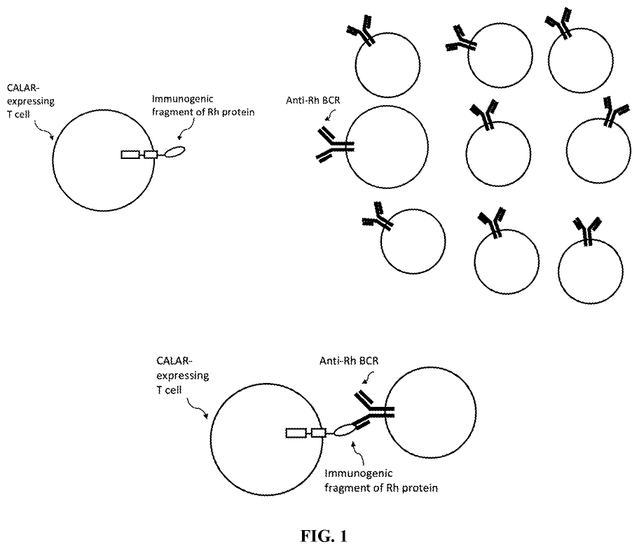

The present disclosure in one aspect relates to the chimeric alloantibody receptors (CALARs) that specifically binds to the alloantibody-based B-cell receptor (BCR) expressed on certain B cells that targets Rh antigen (FIG. 1); after activation, these B cells can produce anti-Rh antibodies, which can cause anti-Rh antibody associated disorder in a fetus after crossing the placental barrier in a pregnant woman. When the CALARs are expressed on an effector cell, such as a T cell, the CALARs specifically direct the effector cells to these B cells, inducing the direct killing of the B cells that express the anti-Rh BCR and the indirect killing of B cells that secrete the anti-Rh antibody, but leaving intact the B cells that do not express and display the anti-Rh BCR or secrete the anti-Rh antibody. Eliminating the pathogenic B cells provides treatment and prevention for disorders associated with maternal anti-Rh antibody, such as hemolysis disease of the newborn (HDN).

In one aspect, the present disclosure provides a CALAR comprising an extracellular domain, a transmembrane domain and an intracellular signaling domain, whereas the extracellular domain comprises an immunogenic fragment of Rh protein.

In another aspect, the present disclosure provides a polynucleotide encoding the CALAR described herein.

### Extracellular Domain

In some embodiments, the extracellular domain of the CALAR described herein comprises an immunogenic fragment of an Rh factor. While the immunogenic fragment is recognized by the alloantibodies against the Rh factor, the immunogenic fragment specifically binds to the BCR of the B-cells that express such alloantibodies.

The immunogenic fragment of the present disclosure can be derived from any Rh factor, for example, RhD or RhCE. The polypeptide and polynucleotide sequences of these factors can be retrieved from public database, such as Uniprot. In certain embodiments, the polypeptide and polynucleotide sequences of these factors are disclosed herein with the sequence identifier in Table 1.

TABLE 1
Sequences of Rh protein.

| | | Polypeptide | Polynucleotide |
|---|---|---|---|
| | | sequence | sequence |
| Rh Protein | Gene Symbol | SEQ ID NO | SEQ ID NO |
| RhD | RHD | 1 | 2 |
| RhCE | RHCE | 3 | 4 |

SEQ ID NO: 1
MSSKYPRSVRRCLPLWALTLEAALILLFYFFTHYDASLEDQKGLVASYQ
VGQDLTVMAAIGLGFLTSSFRRHSWSSVAFNLFMLALGVQWAILLDGFL
SQFPSGKVVITLFSIRLATMSALSVLISVDAVLGKVNLAQLVVMVLVEV
TALGNLRMVISNIFNTDYHMNMMHIYVFAAYFGLSVAWCLPKPLPEGTE
DKDQTATIPSLSAMLGALFLWMFWPSFNSALLRSPIERKNAVFNTYYAV
AVSVVTAISGSSLAHPQGKISKTYVHSAVLAGGVAVGTSCHLIPSPWLA
MVLGLVAGLISVGGAKYLPGCCNRVLGIPHSSIMGYNFSLLGLLGEIIY
IVLLVLDTVGAGNGMIGFQVLLSIGELSLAIVIALMSGLLTGLLLNLKI
WKAPHEAKYFDDQVFWKFPHLAVGF
SEQ ID NO: 2
ATGAGCTCTAAGTACCCGCGGTCTGTCCGGCGCTGCCTGCCCCTCTGGG
CCCTAACACTGGAAGCAGCTCTCATTCTCCTCTTCTATTTTTTTACCCA
CTATGACGCTTCCTTAGAGGATCAAAAGGGGCTCGTGGCATCCTATCAA
GTTGGCCAAGATCTGACCGTGATGGCGGCCATTGGCTTGGGCTTCCTCA
CCTCGAGTTTCCGGAGACACAGCTGGAGCAGTGTGGCCTTCAACCTCTT
CATGCTGGCGCTTGGTGTGCAGTGGGCAATCCTGCTGGACGGCTTCCTG
AGCCAGTTCCCTTCTGGGAAGGTGGTCATCACACTGTTCAGTATTCGGC
TGGCCACCATGAGTGCTTTGTCGGTGCTGATCTCAGTGGATGCTGTCTT
GGGGAAGGTCAACTTGGCGCAGTTGGTGGTGATGGTGCTGGTGGAGGTG
ACAGCTTTAGGCAACCTGAGGATGGTCATCAGTAATATCTTCAACACAG
ACTACCACATGAACATGATGCACATCTACGTGTTCGCAGCCTATTTTGG
GCTGTCTGTGGCCTGGTGCCTGCCAAAGCCTCTACCCGAGGGAACGGAG
GATAAAGATCAGACAGCAACGATACCCAGTTTGTCTGCCATGCTGGGCG
CCCTCTTCTTGTGGATGTTCTGGCCAAGTTTCAACTCTGCTCTGCTGAG
AAGTCCAATCGAAAGGAAGAATGCCGTGTTCAACACCTACTATGCTGTA
GCAGTCAGCGTGGTGACAGCCATCTCAGGGTCATCCTTGGCTCACCCCC
AAGGGAAGATCAGCAAGACTTATGTGCACAGTGCGGTGTTGGCAGGAGG
CGTGGCTGTGGGTACCTCGTGTCACCTGATCCCTTCTCCGTGGCTTGCC
ATGGTGCTGGGTCTTGTGGCTGGGCTGATCTCCGTCGGGGGAGCCAAGT
ACCTGCCGGGGTGTTGTAACCGAGTGCTGGGGATTCCCCACAGCTCCAT
CATGGGCTACAACTTCAGCTTGCTGGGTCTGCTTGGAGAGATCATCTAC
ATTGTGCTGCTGGTGCTTGATACCGTCGGAGCCGGCAATGGCATGATTG
GCTTCCAGGTCCTCCTCAGCATTGGGGAACTCAGCTTGGCCATCGTGAT
AGCTCTCATGTCTGGTCTCCTGACAGGTTTGCTCCTAAATCTTAAAATA
TGGAAAGCACCTCATGAGGCTAAATATTTTGATGACCAAGTTTTCTGGA
AGTTTCCTCATTTGGCTGTTGGATTTTAA
SEQ ID NO: 3
MSSKYPRSVRRCLPLWALTLEAALILLFYFFTHYDASLEDQKGLVASYQ
VGQDLTVMAALGLGFLTSNFRRHSWSSVAFNLFMLALGVQWAILLDGFL
SQFPPGKVVITLFSIRLATMSAMSVLISAGAVLGKVNLAQLVVMVLVEV
TALGTLRMVISNIFNTDYHMNLRHFYVFAAYFGLTVAWCLPKPLPKGTE
DNDQRATIPSLSAMLGALFLWMFWPSVNSPLLRSPIQRKNAMFNTYYAL
AVSVVTAISGSSLAHPQRKISMTYVHSAVLAGGVAVGTSCHLIPSPWLA
MVLGLVAGLISIGGAKCLPVCCNRVLGIHHISVMHSIFSLLGLLGEITY
IVLLVLHTVWNGNGMIGFQVLLSIGELSLAIVIALTSGLLTGLLLNLKI
WKAPHVAKYFDDQVFWKFPHLAVGF
SEQ ID NO: 4
ATGAGCTCTAAGTACCCGCGGTCTGTCCGGCGCTGCCTGCCCCTCTGCG
CCCTAACACTGGAAGCAGCTCTCATTCTCCTCTTCTATTTTTTTACCCA
CTATGACGCTTCCTTAGAGGATCAAAAGGGGCTCGTGGCATCCTATCAA
GTCGGCCAAGATCTGACCGTGATGGCGGCCCTTGGCTTGGGCTTCCTCA
CCTCAAATTTCCGGAGACACAGCTGGAGCAGTGTGGCCTTCAACCTCTT
CATGCTGGCGCTTGGTGTGCAGTGGGCAATCCTGCTGGACGGCTTCCTG
AGCCAGTTCCCTCCTGGGAAGGTGGTCATCACACTGTTCAGTATTCGGC
TGGCCACCATGAGTGCTATGTCGGTGCTGATCTCAGCGGGTGCTGTCTT
GGGGAAGGTCAACTTGGCGCAGTTGGTGGTGATGGTGCTGGTGGAGGTG
ACAGCTTTAGGCACCCTGAGGATGGTCATCAGTAATATCTTCAACACAG
ACTACCACATGAACCTGAGGCACTTCTACGTGTTCGCAGCCTATTTTGG
GCTGACTGTGGCCTGGTGCCTGCCAAAGCCTCTACCCAAGGGAACGGAG
GATAATGATCAGAGAGCAACGATACCCAGTTTGTCTGCCATGCTGGGCG
CCCTCTTCTTGTGGATGTTCTGGCCAAGTGTCAACTCTGCTCTGCTGAG
AAGTCCAATCCAAAGGAAGAATGCCATGTTCAACACCTACTATGCTCTA
GCAGTCAGTGTGGTGACAGCCATCTCAGGGTCATCCTTGGCTCACCCCC
AAAGGAAGATCAGCATGACTTATGTGCACAGTGCGGTGTTGGCAGGAGG
CGTGGCTGTGGGTACCTCGTGTCACCTGATCCCTTCTCCGTGGCTTGCC
ATGGTGCTGGGTCTTGTGGCTGGGCTGATCTCCATCGGGGGAGCCAAGT
GCCTGCCGGTGTGTTGTAACCGAGTGCTGGGGATTCACCACATCTCCGT
CATGCACTCCATCTTCAGCTTGCTGGGTCTGCTTGGAGAGATCACCTAC
ATTGTGCTGCTGGTGCTTCATACTGTCTGGAACGGCAATGGCATGATTG
GCTTCCAGGTCCTCCTCAGCATTGGGGAACTCAGCTTGGCCATCGTGAT
AGCTCTCACGTCTGGTCTCCTGACAGGTTTGCTCCTAAATCTCAAAATA
TGGAAAGCACCTCATGTGGCTAAATATTTTGATGACCAAGTTTTCTGGA
AGTTTCCTCATTTGGCTGTTGGATTTTAA

In certain embodiments, the immunogenic fragment of the Rh factor comprises an epitope of D, C, E, c or e antigen. In certain embodiments, the immunogenic fragment of the Rh factor comprises an epitope of RhD antigen. RhD antigen, accounting for more than 50% of maternal alloimmunization, comprises a collection of over 30 epitopes across the entire Rh D factor. Those RhD epitopes located on the extracellular loops of RhD are majorly responsible for eliciting alloimmunization (Scott M L, Voak D, Jones J W, et al. A structural model for 30 Rh D epitopes based on serological and DNA sequence data from partial D phenotypes. Transfus Clin Biol. 1996, 3:391-396). In certain embodiments, the immunogenic fragment of Rh factor comprises an extracellular loop of RhD protein.

It should be noted that when reference is made to Rh factor or any immunogenic of Rh factor, isoforms or variants comprising amino acid change(s) that minimally affect the recognition by anti-Rh antibody to Rh antigen are also included unless the context dictates otherwise.

Exemplary immunogenic fragments of Rh factor are illustrated in Table 2. In some embodiments, the extracellular domain of the CALAR comprises one or more sequences selected from the group of sequences listed in Table 2, or one or more sequences having at least 80%, 85%, 90%, 91%, 92%, 93%, 94%, 95%, 96%, 97%, 98%, 99% sequence identity thereto, or one or more sequences having 1, 2, 3, 4, or 5 amino acid residue difference therefrom.

TABLE 2
Immunogenic fragment of Rh factor.

| | SEQ ID | | Gene | |
|---|---|---|---|---|
| Sequence | NO | Rh factor | Symbol | Location |
| HYDASLEDQKGLVASYQVGQDLT | 5 | D, C, c, | RHD, RHCE | extracellular loop |
| | | E, e | | 1 |
| FPSGKVVITLFSIRL | 6 | D, C | RHD, RHCE | extracellular loop |
| | | | | 2 |
| NIFNTDYHMNMMHI | 7 | D | RHD | extracellular loop |
| | | | | 3 |
| WPSFNSALLRSPIERKNAVFN | 8 | D | RHD | extracellular loop |
| | | | | 4 |
| CHLIPS | 9 | D, C, c, | RHD, RHCE | extracellular loop |
| | | E, e | | 5 |
| VLDTVGAGNGM | 10 | D | RHD | extracellular loop |
| | | | | 6 |
| FPPGKVVITLFSIRL | 11 | C | RHCE | extracellular loop |
| | | | | 2 |
| NIFNTDYHMNLRHF | 12 | C, c, E, e | RHCE | extracellular loop |
| | | | | 3 |
| WPSVNSALLRSPIQRKNAMFN | 13 | e | RHCE | extracellular loop |
| | | | | 4 |
| WPSVNSPLLRSPIQRKNAMFN | 14 | E | RHCE | extracellular loop |
| | | | | 4 |
| VLHTVWNGNGM | 15 | C, c, E, e | RHCE | extracellular loop |
| | | | | 6 |

In some embodiments, the extracellular domain of the CALAR described herein further comprises a signal peptide. The term “signal peptide” as used herein refers to peptide, usually having a length of 5-30 amino acid residues, present at the N-terminus of a polypeptide that necessary for the translocation cross the membrane on the secretory pathway and control of the entry of the polypeptide to the secretory pathway.

In some embodiments, the signal peptide comprises a signal peptide of CD8 alpha. In some embodiments, the signal peptide of CD8 alpha comprises a sequence of SEQ ID NO: 16 or a sequence having at least 80%, 85%, 90%, 91%, 92%, 93%, 94%, 95%, 96%, 97%, 98%, 99% sequence identity thereto. In some embodiments, the signal peptide comprises a signal peptide of IgG.

### Transmembrane Domain

The transmembrane domain of the CALAR described herein may be derived from any membrane-bound or transmembrane protein including, but are not limited to, BAFFR, BLAME (SLAMF8), CD2, CD3 epsilon, CD4, CD5, CD8, CD9, CD11a (CD18, ITGAL, LFA-1), CD11b, CD11c, CD11d, CD16, CD19, CD22, CD27, CD28, CD29, CD33, CD37, CD40, CD45, CD49a, CD49d, CD49f, CD64, CD80, CD84, CD86, CD96 (Tactile), CD100 (SEMA4D), CD103, CD134, CD137 (4-1BB), CD150 (IPO-3, SLAMF1, SLAM), CD154, CD160 (BY55), CD162 (SELPLG), CD226 (DNAM1), CD229 (Ly9), CD244 (2B4, SLAMF4), CD278 (ICOS), CEACAM1, CRT AM, GITR, HYEM (LIGHTR), IA4, IL2R beta, IL2R gamma, IL7R a, ITGA1, ITGA4, ITGA6, ITGAD, ITGAE, ITGAM, ITGAX, ITGB1, ITGB2, ITGB7, KIR, LTBR, OX40, NKG2C, NKG2D, NKp30, NKp44, NKp46, NKp80 (KLRF1), PAG/Cbp, PSGL1, SLAMF6 (NTB-A, Ly108), SLAMF7, an alpha, beta or zeta chain of a T-cell receptor, TNFR2, VLA1, and VLA-6.

In one embodiment, the CALAR described herein comprises a transmembrane domain of CD8 alpha, CD28 or ICOS. In certain embodiments, the transmembrane domain of CD8 alpha has a sequence of SEQ ID NO: 17, or a sequence having at least 80%, 85%, 90%, 91%, 92%, 93%, 94%, 95%, 96%, 97%, 98%, 99% sequence identity thereto, or a sequence having 1, 2, 3, 4 or 5 amino acid residues difference therefrom.

In certain embodiments, the transmembrane domain of the CALAR described herein is synthetic, e.g., comprising predominantly hydrophobic residues such as leucine and valine. In certain embodiments, the transmembrane domain of the CALAR described herein is modified or designed to avoid binding to the transmembrane domains of the same or different surface membrane proteins in order to minimize interactions with other members of the receptor complex.

In some embodiments, the CALAR described herein further comprises a hinge region, which forms the linkage between the extracellular domain and transmembrane domain of the CALAR. The hinge and/or transmembrane domain provides cell surface presentation of the extracellular domain of the CALAR.

The hinge region may be derived from any membrane-bound or transmembrane protein including, but are not limited to, BAFFR, BLAME (SLAMF8), CD2, CD3 epsilon, CD4, CD5, CD8, CD9, CD11a (CD18, ITGAL, LFA-1), CD11b, CD11c, CD11d, CD16, CD19, CD22, CD27, CD28, CD29, CD33, CD37, CD40, CD45, CD49a, CD49d, CD49f, CD64, CD80, CD84, CD86, CD96 (Tactile), CD100 (SEMA4D), CD103, CD134, CD137 (4-1BB), CD150 (IPO-3, SLAMF1, SLAM), CD154, CD160 (BY55), CD162 (SELPLG), CD226 (DNAM1), CD229 (Ly9), CD244 (2B4, SLAMF4), CD278 (ICOS), CEACAM1, CRT AM, GITR, HYEM (LIGHTR), IA4, IL2R beta, IL2R gamma, IL7Ra, ITGA1, ITGA4, ITGA6, ITGAD, ITGAE, ITGAM, ITGAX, ITGB1, ITGB2, ITGB7, KIR, LTBR, OX40, NKG2C, NKG2D, NKp30, NKp44, NKp46, NKp80 (KLRF1), PAG/Cbp, PSGL1, SLAMF6 (NTB-A, Ly108), SLAMF7, an alpha, beta or zeta chain of a T-cell receptor, TNFR2, VLA1, and VLA-6.

In some embodiments, the hinge region comprises a hinge region of CD8 alpha, a hinge region of human immunoglobulin (Ig), or a glycine-serine rich sequence.

In some embodiments, the CALAR comprises a hinge region of CD8 alpha, CD28, ICOS or IgG4m. In certain embodiments, the hinge region has a sequence of SEQ ID NO: 18, or a sequence having at least 80%, 85%, 90%, 91%, 92%, 93%, 94%, 95%, 96%, 97%, 98%, 99% sequence identity thereto, or a sequence having 1, 2, 3, 4 or 5 amino acid residues difference therefrom.

In some embodiments, the hinge region comprises a GS linker comprising glycine-serine (GS) doublet between 2 and 20 amino acid residues in length. Exemplary GS GS linker has the sequence of SEQ ID NO: 19.

### Intracellular Domain

The intracellular domain of the CALAR described herein, is responsible for activation of at least one of the normal effector functions of the immune cell in which the CALAR has been placed in. The term “effector function” used in the context of an immune cell refers to a specialized function of the cell, for example, the cytolytic activity or helper activity including the secretion of cytokines for a T cell.

It is well recognized that the full activation of a T-cell requires signals generated through the T-cell receptor (TCR) and a secondary or co-stimulatory signal. Thus, the T cell activation is mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

The intracellular domain of the CALAR can be derived from a molecule which transduces the effector function signal and directs the cell to perform the effector function, or a truncated portion of such molecule as long as it transduces the signal. Such protein molecule including, but are not limited to, B7-H3, BAFFR, BLAME (SLAMF8), CD2, CD3 delta, CD3 epsilon, CD3 gamma, CD3 zeta, CD4, CD5, CD7, CD8alpha, CD8beta, CD11a (CD18, LFA-1, ITGAL), CD11b, CD11c, CD11d, CD19, CD27, CD28, CD29, CD30, CD40, CD49a, CD49d, CD49f, CD69, CD79a, CD79b, CD83, CD84, CD86, CD96 (Tactile), CD100 (SEMA4D), CD103, CD127, CD137 (4-1BB), CD150 (SLAM, SLAMF1, IPO-3), CD160 (BY55), CD162 (SELPLG), CD226 (DNAM1), CD229 (Ly9), CD244 (SLAMF4, 2B4), CEACAM1, CRTAM, DAP10, DAP12, common FcR gamma, FcR beta (Fc Epsilon Rib), Fcgamma RIIa, GADS, GITR, HVEM (LIGHTR), IA4, IL2R beta, IL2R gamma, IL7R alpha, ITGA4, ITGA6, ITGAD, ITGAE, ITGAM, ITGAX, ITGB1, ITGB2, ITGB7, ICAM-1, ICOS, LIGHT, LTBR, LAT, NKG2C, NKG2D, NKp44, NKp30, NKp46, NKp80 (KLRF1), OX40, PD-1, PAG/Cbp, PSGL1, SLP-76, SLAMF6 (NTB-A, Ly108), SLAMF7, T cell receptor (TCR), TNFR2, TRANCE/RANKL, VLA1, VLA-6, any derivative, variant, or fragment thereof, any synthetic sequence of a molecule that has the same functional capability, and any combination thereof.

In some embodiments, the intracellular domain comprises a co-stimulatory domain and a signaling domain, wherein upon binding of the CALAR to the BCR expressed on a B cell that is specific for Rh antigen, the co-stimulatory domain provides co-stimulatory intracellular signaling without the need of its original ligand, and the signaling domain provides the primary activation signaling. The co-stimulatory domain and the signaling domain of the CALAR can be linked to each other in a random or specified order.

### Co-Stimulatory Domain

In some embodiments, the co-stimulatory domain is derived from an intracellular domain of a co-stimulatory molecule.

Examples of co-stimulatory molecules include B7-H3, BAFFR, BLAME (SLAMF8), CD2, CD4, CD8 alpha, CD8 beta, CD7, CD11a, CD11b, CD11c, CD11d, CD 18, CD 19, CD27, CD28, CD29, CD30, CD40, CD49a, CD49D, CD49f, CD69, CD83, CD84, CD96 (Tactile), CD100 (SEMA4D), CD103, CD 127, CD137(4-1BB), CD150 (SLAM, SLAMF1, IPO-3), CD160 (BY55), CD162 (SELPLG), CD226 (DNAM1), CD229 (Ly9), CD244 (SLAMF4, 2B4), CEACAM1, CRTAM, CDS, OX40, PD-1, ICOS, GADS, GITR, HVEM (LIGHTR), IA4, ICAM-1, IL2R beta, IL2R gamma, IL7R alpha, ITGA4, ITGA4, ITGA6, ITGAD, ITGAE, ITGAL, ITGAM, ITGAX, ITGB1, ITGB2, ITGB7, LAT, LFA-1, LIGHT, LTBR, NKG2C, NKG2D, NKp44, NKp30, NKp46, NKp80 (KLRF1), PAG/Cbp, PSGL1, SLAMF6 (NTB-A, Ly108), SLAMF7, SLP-76, TNFR2, TRANCE/RANKL, VLA1, VLA-6, any derivative, variant, or fragment thereof, any synthetic sequence of a co-stimulatory molecule that has the same functional capability, and any combination thereof.

In some embodiment, the co-stimulatory domain of the CALAR comprises an intracellular domain of co-stimulatory molecule CD137 (4-1BB), CD28, OX40 or ICOS. In some embodiments, the co-stimulatory domain of the CALAR has a sequence of SEQ ID NO: 20. or a sequence having at least 80%, 85%, 90%, 91%, 92%, 93%, 94%, 95%, 96%, 97%, 98%, 99% sequence identity thereto, or a sequence having 1, 2, 3, 4 or 5 amino acid residues difference therefrom.

### Signaling Domain

The primary activation of the TCR complex can be regulated by a primary cytoplasmic signaling sequence either in a stimulatory manner or in an inhibitory manner. Primary cytoplasmic signaling sequences that act in a stimulatory manner may contain signaling motifs known as immunoreceptor tyrosine-based activation motifs (ITAMs). Examples of ITAM containing primary signaling sequences that are of particular use in the disclosure include those derived from CD3 gamma, CD3 delta, CD3 epsilon, CD3 zata, CDS, CD22, CD79a, CD79b, CD66d, FcR gamma, FcR beta, and TCR zeta.

In some embodiments, the signaling domain of the CALAR of the disclosure comprises an ITAM that provides stimulatory intracellular signaling upon the CALAR binding to BCR expressed on a B cell that is specific for Rh antigen, without HLA restriction. In some embodiments, the signaling domain of the CALAR comprises a signaling domain of CD3 zeta (CD247). In some embodiments, the signaling domain of the CALAR comprises a sequence of SEQ ID NO: 21, or a sequence having at least 80%, 85%, 90%, 91%, 92%, 93%, 94%, 95%, 96%, 97%, 98%, 99% sequence identity thereto, or a sequence having 1, 2, 3, 4 or amino acid residues difference therefrom.

### Other Region

In some embodiments, the CALAR further comprises a linker. The term “linker” as provided herein is a polypeptide connecting various components of the CALAR.

In some embodiments, the linker is inserted between the transmembrane domain and the intracellular domain. In some embodiments, the linker is between the signaling domain and the co-stimulatory domain of the intracellular domain.

In some embodiments, the linker is a GS linker comprising glycine-serine (GS) doublet between 2 and 20 amino acid residues in length. Exemplary GS linker has the sequence shown in SEQ ID NO: 19. In some embodiments, the polynucleotide provided herein comprises a nucleotide sequence encoding a linker.

In some embodiments, the CALAR provided herein comprises from the N-terminus to the C-terminus: a signal peptide of CD8 alpha, an immunogenic fragment of Rh protein (e.g., a sequence selected from Table 2 or any combination thereof), a GS linker, a transmembrane domain of CD8 alpha, an intracellular domain of CD137, and a signaling domain of CD3 zeta.

In some embodiments, the polynucleotide provided herein encodes a CALAR comprising from the N-terminus to the C-terminus: a signal peptide of CD8 alpha, an immunogenic fragment of Rh factor (e.g., a sequence selected from Table 2 or any combination thereof), a GS linker, a transmembrane domain of CD8 alpha, an intracellular domain of CD137, and a signaling domain of CD3 zeta.

In some embodiments, the CALAR demonstrates a high affinity to an anti-Rh antibody. The term “affinity” as used herein refers to the strength of non-covalent interaction between an immunoglobulin molecule or fragment thereof and an antigen. The binding affinity can be represented by Kd value, i.e., the dissociation constant, determined by any methods known in the art, including, without limitation, enzyme-linked immunosorbent assays (ELISA), surface plasmon resonance, or flow cytometry (such as FACS). In certain embodiments, the CALAR has a binding affinity to anti-Rh antibody of less than 50 nM, nM, 10 nM, 5 nM, 4 nM, 3 nM, 2 nM, or 1 nM.

### Vector

In another aspect, the present disclosure provides a vector comprising the polynucleotide encoding the CALAR as described herein. The polynucleotides encoding a CALAR can be inserted into different types of vectors known in the art, for example, a plasmid, a phagemid, a phage derivative, a viral vector derived from animal virus, a cosmid, transposon, a site directed insertion vector (e.g., CRISPR, Zinc finger nucleases, TALEN), or a suicide expression vector. In some embodiments, the vector is a DNA or RNA.

In some embodiment, the polynucleotide is operatively linked to at least one regulatory polynucleotide element in the vector for expression of the CALAR. Typical vectors contain various regulatory polynucleotide elements, for example, elements (e.g., transcription and translation terminators, initiation sequences, and promoters) regulating the expression of the inserted polynucleotides, elements (e.g., origin of replication) regulating the replication of the vector in a host cell, and elements (e.g., terminal repeat sequence of a transposon) regulating the integration of the vector into a host genome. The expression of the CALAR can be achieved by operably linking the polynucleotides encoding a CALAR to a promoter, and incorporating the construct into a vector. Both constitutive promoters (such as a CMV promoter, a SV40 promoter, and a MMTV promoter), or inducible promoters (such as a metallothionine promoter, a glucocorticoid promoter, and a progesterone promoter) are contemplated for the disclosure. In some embodiment, the vector is an expression vector. An expression vector comprises sufficient cis-acting elements for expression; other elements for expression can be supplied by the host cell or in an in vitro expression system.

In order to assess the expression of a CALAR, the vector can also comprise a selectable marker gene or a reporter gene or both for identification and selection of the cells to which the vector are introduced. Useful selectable markers include, for example, antibiotic-resistance genes, such as neo and the like. Useful reporters include, for example, luciferase, beta-galactosidase, chloramphenicol acetyl transferase, secreted alkaline phosphatase, or the green fluorescent protein gene.

In some embodiments, the vector is a viral vector. Viral vectors may be derived from, for example, retroviruses, adenoviruses, adeno-associated viruses (AAV), herpes viruses, and lentiviruses. Useful viral vectors generally contain an origin of replication functional in at least one organism, a promoter, restriction endonuclease sites, and one or more selectable markers. In some embodiments, the vector is a retrovirus vector, such as lentiviral vector. Lentiviral vector is particular useful for long-term, stable integration of the polynucleotide encoding the CALAR into the genome of non-proliferating cells that result in stable expression of the CALAR in the host cell, e.g., host T cell.

In some embodiments, the vector is mRNA, which can be electroporated into the host cell. As the mRNA would dilute out with cell division, the expression of the mRNA would not be permanent.

In some embodiments, the vector is a transposon-based expression vector. A transposon is a DNA sequence that can change its position within a genome. In a transposon system, the polynucleotide encoding the CALAR is flanked by terminal repeat sequences recognizable by a transposase which mediates the movement of the transposon. A transposase can be co-delivered as a protein, encoded on the same vector as the CALAR, or encoded on a separate vector. Non-limiting examples of transposon systems include Sleeping Beauty, Piggyback, Frog Prince, and Prince Charming.

A vector can be introduced into a host cell, e.g., mammalian cell by any method known in the art, for example, by physical, chemical or biological means. Physical methods for introducing a polynucleotide into a host cell include calcium phosphate precipitation, lipofection, particle bombardment, microinjection, electroporation, and the like. Biological methods include the use of viral vectors, and especially retroviral vectors, for inserting genes into mammalian, e.g., human cells. Chemical means include colloidal dispersion systems, such as macromolecule complexes, nanocapsules, microspheres, beads, and lipid-based systems including oil-in-water emulsions, micelles, mixed micelles, and liposomes.

### Cells

In one aspect, the disclosure provides an engineered cell comprising or expressing the CALAR as described here. In some embodiments, the engineered cell comprises the polynucleotide encoding the CALAR, or the vector comprising the CALAR polynucleotide. In some embodiments, an engineered cell comprises multiple CALAR comprising different immunogenic fragments of Rh factor.

An engineered cell as described herein is a genetically modified immune cell, Immune cells useful for the disclosure include T cells, natural killer (NK) cells, invariant NK cells, or NKT cells, and other effector cell. In some embodiment, the immune cells are primary cells, expanded cells derived from primary cells, or cells derived from stem cells differentiated in vitro.

It is useful for an engineered cell comprising or expressing a CALAR to have high affinity for an alloantibody-based BCR expressed by B cells, wherein the alloantibody specifically binds Rh factor or an immunogenic fragment thereof. As a result, the engineered cell can induce direct killing of anti-Rh B cells or indirect killing of plasma cells expressing the anti-Rh alloantibodies. In some embodiments, the engineered cell has low affinity for alloantibodies to Rh that are bound to an Fc receptor.

In another aspect, the disclosure provides a method of making an engineered cell expressing the CALAR as described herein. In some embodiments, the method comprising one of more steps selected from of obtaining cells from a source, culturing cells, activating cells, expanding cells and engineering cells with a vector comprising the polynucleotide of a CALAR.

In another aspect, the disclosure provides a method of using the engineered cells for cell therapy, wherein the engineered cells are introduced into a subject. In some embodiments, the subject is the same subject from who the cells are obtained (autologous cells).

### Sources of Cells

The engineered cells can be derived from immune cells isolated from subjects, e.g., human subjects. In some embodiments, the immune cells are obtained from a subject of interest, such as a subject suspected of having a particular disease or condition, a subject suspected of having a predisposition to a particular disease or condition, a subject who will undergo, is undergoing, or have undergone treatment for a particular disease or condition, a subject who is a healthy volunteer or healthy donor, or from blood bank. Thus, the cells can be autologous or allogeneic to the subject of interest. Allogeneic donor cells may not be human-leukocyte-antigen (HLA)-compatible, and thus allogeneic cells can be treated to reduce immunogenicity.

Immune cells can be collected from any location in which they reside in the subject including, but not limited to, blood, cord blood, spleen, thymus, lymph nodes, pleural effusion, spleen tissue, and bone marrow. The isolated immune cells may be used directly, or they can be stored for a period of time, such as by freezing.

In some embodiments, the engineered cells are derived from T cells. T cells can be obtained from blood collected from a subject using any number of techniques known to the skilled artisan, such as apheresis.

In some embodiments, one or more of the T cell populations is enriched for or depleted of cells that are positive, or negative, for a specific marker, such as surface markers. Such markers are those that are absent or expressed at relatively low levels on certain populations of T cells but are present or expressed at relatively higher levels on certain other populations of T cells. In some embodiments, CD4+ helper and CD8+ cytotoxic T cells are isolated. In some embodiments, CD8+ and CD4+ T cells are further enriched for or depleted of naive, central memory, effector memory, and/or central memory stem cells, such as by positive or negative selection based on surface antigens associated with the respective subpopulation.

### Activation and Expansion of Cells

In some embodiments, the immune cells are activated and expanded prior to genetically modification. In other embodiments, the immune cells are activated, but not expanded, or are neither activated nor expanded prior to use.

Method for activation and expansion of immune cells have been described in the art and can be used in the methods described herein. For example, the T cells can be activated and expanded by contacting with a surface having attached thereto an agent that stimulates a CD3/TCR complex associated signal and a ligand that stimulates a co-stimulatory molecule on the surface of the T cells. To stimulate proliferation of either CD4+ T cells or CD8+ T cells, an anti-CD3 antibody and an anti-CD28 antibody can be used.

### Method of Treatment

In one aspect, the present disclosure provides a method of treating or preventing a disorder associated with anti-Rh antibody comprising an effective amount of the engineered cell described herein in a subject in need thereof. In some embodiments, the disorder associated with anti-Rh antibody is hemolysis disease of the newborn. In some embodiments, the disorder associated with Rh alloantibody is delayed hemolytic transfusion reactions (DHTRs). In some embodiments, the subject in need thereof is a woman having anti-Rh antibody in her blood.

In some embodiments, the engineered cell comprising or expressing a CALAR is derived from T cells isolated from a subject, expanded ex vivo, engineered to comprise a vector for expressing the CALAR, and transfused into the subject. The engineered cells (e.g. T cells) recognize B cells expressing and presenting an alloantibody-based BCR, wherein the alloantibody specifically targets Rh factor, and the engineered cells become activated, resulting in killing of the targeted B cells. In some embodiments, the engineered cells (e.g. T cells) are autologous cell.

In certain embodiments, the treatment method further comprises administering an agent that increases the efficacy of the engineered cells. For example, a growth factor that promotes the growth and activation of the engineered cells of the present disclosure is administered to the subject either concomitantly with the cells or subsequently to the cells. The growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

In some embodiments, the treatment method further comprises administering an agent that reduces of ameliorates a side effect associated with the administration of the engineered cells. Exemplary side effects include cytokine response syndrome (CRS), and hemophagocytic lymphohistiocytosis (HLH, also termed macrophage activation syndrome (MAS)). The agent administered to treat the side effects can be an agent neutralizing soluble factors such as IFN-gamma, IFN-alpha, IL-2 and IL-6. Such agents include, without limitation, an inhibitor of TNF-alpha (e.g., entanercept) and an inhibitor of IL-6 (e.g., tocilizumab).

Therapeutically effective amounts of the engineered cells can be administered by a number of routes, including parenteral administration, for example, intravenous, intraperitoneal, intramuscular, intrasternal, or intraarticular injection, or infusion.

The engineered cells can be administered in treatment regimens consistent with the titer of the anti-Rh antibody in the subject in need thereof, for example a single or a few doses over one to several days or periodic doses over an extended time. The precise dose to be employed in the formulation will also depend on the route of administration, and should be decided according to the judgment of the practitioner and each patient's circumstances. The therapeutically effective amount of engineered cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ cells/m2. In a certain embodiment, the dose used in the treatment of human subjects ranges from about 3.8×10⁹ to about 3.8×10¹⁰ cells/m2. In additional embodiments, a therapeutically effective amount of the engineered cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of engineered cells is readily determined by one of skill in the art based on the age, weight, sex, and physiological condition of the subject. Effective doses can be extrapolated from dose-response curves derived from in vitro or animal model test systems.

In some embodiments, the engineered cell comprising a CALAR can be administered before, during, following, or in any combination relative to an additional pharmaceutical agent for the treatment or prevention.

In another aspect, the present disclosure also provides a pharmaceutical composition comprising the engineered cells and a pharmaceutically acceptable diluent and/or carrier. Exemplary diluent and/or carrier include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

TABLE 3
Exemplary sequences of domains comprised in CALAR

| SEQ ID NO. | Sequence | Domain |
|---|---|---|
| 16 | MALPVTALLLPLALLLHAARP | CD8 alpha signal |
| | | peptide |
| 17 | IYIWAPLAGTCGVLLLSLVIT | CD8 alpha |
| | | transmembrane domain |
| 18 | TTTPAPRPPTPAPTIASQPLSLRPEACRPAA | CD8 alpha hinge |
| | GGAVHTRGLDFACD | region |
| 19 | GGGGSGGGGSSG | GS linker |
| 20 | KRGRKKLLYIFKQPFMRPVQTTQEEDGCSC | CD137 intracellular |
| | RFPEEEEGGCEL | domain |
| 21 | RVKFSRSADAPAYQQGQNQLYNELNLGRR | CD3 zeta (CD247) |
| | EEYDVLDKRRGRDPEMGGKPRRKNPQEGL | signaling domain |
| | YNELQKDKMAEAYSEIGMKGERRRGKGH | |
| | DGLYQGLSTATKDTYDALHMQALPPR | |

### EXAMPLE

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1

This example illustrates the efficacy of CALAR T cells in vitro.

Design and construction of RHD CALAR. Two RHD CALAR constructs, named RHD1 (amino acid sequence of SEQ ID NO: 22) and RHD2 (amino acid sequence of SEQ ID NO: 23) were designed and synthesized by Genewiz (Suzhou, China). The RHD1 and RHD2 genes were cloned into a third-generation lentiviral vector.

Lentiviral production. VSV-G pseudotyped lentiviral particles were produced using a third-generation packaging system. 293T cells were transfected at a confluency of 80% with a mixture of the transfer plasmid, the envelope plasmid, the packaging plasmids, and Lipofectamine 3000 (Life Technologies). Lentivirus containing supernatant was harvested after 48 hours, filtered through a 0.45 micron PVDF membrane, concentrated at 1500×g for 45 min at 4° C. and stored at −80° C.

Activation and expansion of primary human T cells. Human PBMC from healthy donor were activated with CD3/CD28 Dynabeads (Thermo Fisher Scientific) at a 1:1 cell/bead ratio for 24 hours. 2×10⁶ T cells were transduced with virus particles. T cells were cultured in XF T Cell Expansion Medium (STEMCELL Technologies) supplemented with 400 U/ml IL-2. Media was changed every 2 to 3 days. Five days after stimulation, positive CAR-T cells were validated by flow cytometry (Beckman cytoflex).

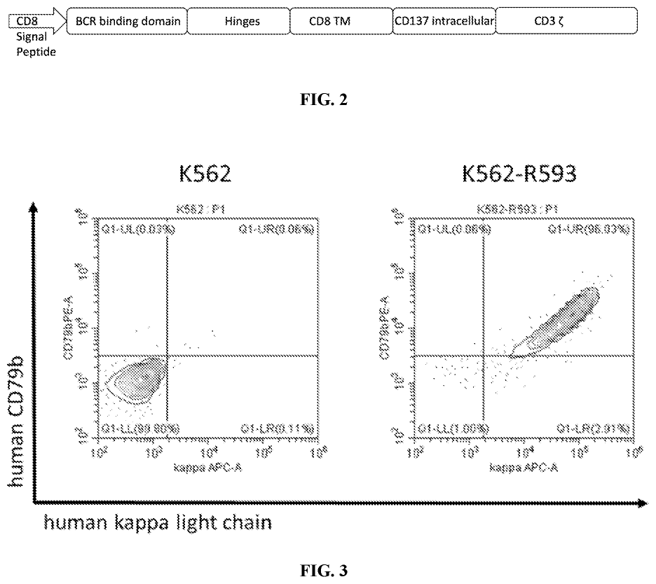

Anti-RhD-specific target cell production. In order to test the killing function of CAR-T in vitro, a target cell that expresses anti-Rh BCR was engineered. There are two steps for the generation of the target cell. In the first step, K562 cells (ATCC: CCL-243) were engineered to express surface IgG BCRs, CD79a and CD79b co-receptors by means of lentiviral transduction. In the second step, CD79a/b positive cells were transduced with lentiviral vectors for surface expression of IgG of the human anti-RhD clones R593. The specific target cell identity was confirmed by flow cytometry and named as K562-R593 (FIG. 3).

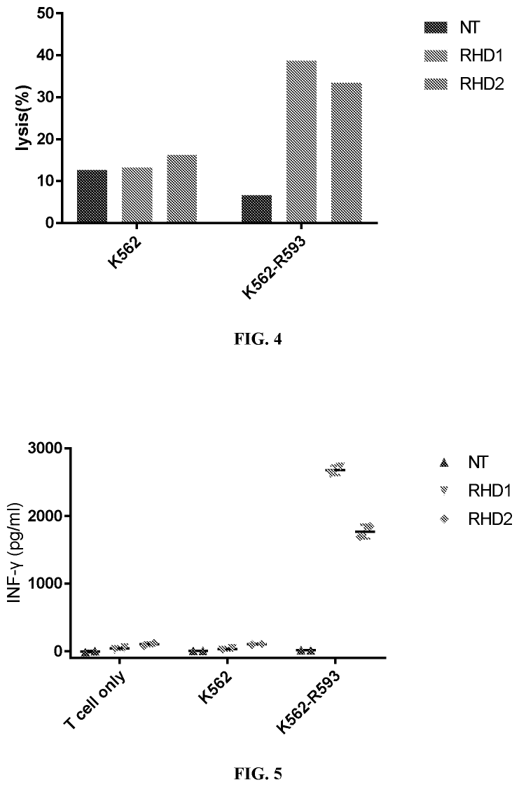

In vitro efficacy testing of CALAR T cell. CALAR T cells (or control T cells) are co-incubated with cells that express anti-Rh BCRs (target cells). The target cells, such as hybridoma cells, is generated in-house. Cytotoxicity is calculated based on percent lysis of target cells. CALAR T cells specific kills target cells but not non-target cells, indicating specificity of target cell killing by the CALAR T cells. K562 and K562-R593 target cells were stained first with CFSE (1 μM) for 5 minutes at 37° C., washed twice and resuspended in X-VIVO15 Medium supplemented with 400 IU/ml IL-2 and 2% SR (Serum Replacement). RHD1 CALAR, RHD2 CALAR, or non-transduced T cells (8-10 days after initial activation) were co-incubated with loaded target cells for 20 hours at 1:1 effector:target (E:T) ratios. Subsequently, 200 microliters of cell suspension were taken and counted directly by flow cytometry. As shown in FIG. 4, CALAR T cells (RHD1 and RHD2) specifically killed K562-R593 target cells but not K562 non-target cells, indicating the specificity of target cell killing by the CALAR T cells. Lysis (%)=[(only target CFSE+cell number−co-incubated CFSE+cell number)/only target CFSE+cell number]*100. INF-γ production of the CALAR T cells was quantified by ELISA according to the manufacturer's recommendations after co-culture at 1:1 effector:target (E:T) ratios in 200 μl for 20 hours. As shown in FIG. 5, CALAR T cells generated increased amount of INF-γ after co-cultured with the K562-R593 target cells but not K562 non-target cells.

### Example 2

This example illustrates the efficacy of CALAR T cells in vivo.

In vivo efficacy testing of CALAR T cell. K562-R593 target cells (generated as in Example 1) or K562 cells as control are injected intravenously into NSG mice after pre-treatment of mice with intravenous immunoglobulin to minimize FcyR-mediated toxicity against BCR-expressing cells. After a few days, RHD CALAR T cells (or control T cells) are injected intravenously. Bioluminescence and/or serum antibodies to Rh are quantified to monitor RHD CALAR T cell efficacy. The results show that RHD CALAR T cells control the growth of K562-R593 target cells but not K562 non-target cells, whereas negative control T cells do not control the outgrowth of K562-R593 target cells.

1. A polynucleotide encoding a chimeric alloantibody receptor (CALAR), wherein the CALAR comprising an extracellular domain comprising an immunogenic fragment of an Rh factor, a transmembrane domain and an intracellular signaling domain, wherein the extracellular domain binds to a B cell receptor (BCR) to a Rh antigen expressed on a B-cell, wherein a cell expressing the CALAR binds the BCR expressed on the B-cell or induces killing of the B-cell expressing the BCR.

2. The polynucleotide of claim 1, wherein the immunogenic fragment of the Rh factor comprises an epitope of Rh antigen.

3. The polynucleotide of claim 1, wherein the immunogenic fragment of Rh factor comprises a sequence selected from the group listed in Table 2 or a sequence having at least 90% identity thereto, or a sequence having 1, 2, 3, 4 or 5 amino acid residues difference therefrom.

4. The polynucleotide of claim 1, wherein the CALAR further comprises a signal peptide.

5. The polynucleotide of claim 4, wherein the signal peptide is the signal peptide of CD8 alpha comprising the sequence of SEQ ID NO: 16 or a sequence having at least 90% identity thereto; or a sequence having 1, 2, 3, 4, 5 amino acid residue difference therefrom.

6. The polynucleotide of claim 1, wherein the transmembrane domain comprises a transmembrane domain of CD8 alpha.

7. The polynucleotide of claim 6, wherein the transmembrane domain of CD8 alpha comprises the sequence of SEQ ID NO: 17 or a sequence having at least 90% identity thereto; or a sequence having 1, 2, 3, 4, 5 amino acid residue difference therefrom.

8. The polynucleotide of claim 1, wherein the extracellular domain is linked to the transmembrane domain by a hinge region.

9. The polynucleotide of claim 8, wherein the hinge region comprises a GS linker or a hinge region of CD8 alpha.

10. The polynucleotide of claim 9, wherein the OS linker comprises the sequence of SEQ ID NO: 19 or a sequence having at least 90% identity thereto; or a sequence having 1, 2, 3, 4, 5 amino acid residue difference therefrom.

11. The polynucleotide of claim 1, wherein the intracellular domain comprises a costimulatory domain and a signaling domain.

12. The polynucleotide of claim 11, wherein the costimulatory domain comprises an intracellular domain of CD137.

13. The polynucleotide of claim 12, wherein the intracellular domain of CD137 comprises the sequence of SEQ ID NO: 20 or a sequence having at least 90% identity thereto; or a sequence having 1, 2, 3, 4, 5 amino acid residue difference therefrom.

14. The polynucleotide of claim 11, wherein the intracellular domain comprises a signaling domain of CD3 zeta.

15. The polynucleotide of claim 14, wherein the signaling domain of CD3 zeta comprises the sequence of SEQ ID NO: 21 or a sequence having at least 90% identity thereto; or a sequence having 1, 3, 4, 5 amino acid residue difference therefrom.

16. A polypeptide encoded by the polynucleotide of claim 1.

17. A vector comprising the polynucleotide of claim 1, wherein the polynucleotide encoding the CALAR is operatively linked to at least one regulatory polynucleotide element for expression of the CALAR.

18. The vector of claim 17, wherein the vector is a plasmid vector, a viral vector, a retrotransposon, a site directed insertion vector, a lentiviral vector, a retroviral vector or an AAV vector or a suicide expression vector.

19. (canceled)

20. An engineered cell comprising the polynucleotide of claim 1.

21. (canceled)

22. A method of treating or preventing a disorder associated with Rh alloantibody, comprising administering an effective amount of the engineered cell of claim 20 in a subject in need thereof.

23-28. (canceled)
